(19) United States
(12) Patent Application Publication
(10) Pub. No.: US 2016/0022694 A1
(43) Pub. Date: Jan. 28, 2016

(54) A Method of Improving the Efficiency of Beef Production from Bovine Animals

(71) Applicants: INTERVET INTERNATIONAL B.V., AN Boxmeer (NL); INTERVET INC., Summit, NJ (US)

(72) Inventors: John Hutcheson, Amarillo, TX (US); David Yates, Claremore, OK (US); Mary Wray, Olathe, KS (US); John Graettinger, Baldwin City, KS (US)

(73) Assignee: Intervet Inc., Madison, NJ (US)

(21) Appl. No.: 14/776,024

(22) PCT Filed: Mar. 14, 2014
(86) PCT No.: PCT/EP2014/055051

§ 371 Date: Sep. 14, 2015

Related U.S. Application Data
(60) Provisional application No. 61/792,511, filed on Mar. 15, 2013.

(51) Int. Cl. A61K 31/55; A23K 1/18; A23K 1/14; A23K 1/16

(52) U.S. Cl. CPC A61K 31/55; A23K 1/1628; A23K 1/1813; A23K 1/146

(57) ABSTRACT

A method of improving the efficiency of beef production from a bovine animal which is fed multiple times per day, comprising delivering to the animal an effective dose of zilpaterol in one or more but not all of daily feedings and wherein the zilpaterol is thoroughly mixed into the feed before use.

### BACKGROUND OF THE INVENTION

In intensive production systems, bovine animals such as cattle, are fed in environments with limited areas, such as in stalls, pens and feedlots in high stocking densities.

Ruminant animals, such as beef cattle, are classified as herbivores, meaning they can survive and produce while feeding chiefly on grass or other roughage feed ingredients consisting of large amounts of cellulose. However, cattle which are being intensively produced for slaughter will normally be placed in a confined feeding facility (feedlot) at 7-15 months of age, and fed growing diets consisting of 30-60% roughage and/or finishing diets consisting of only 5-15% roughage (conventional finishing ration on a dry basis), the roughage will normally be in the form of hay, silage, fodder, corn cobs, cottonseed hulls, etc. The remainder of the diet will consist of a high energy grain source such as corn, grain sorghum, barley, wheat, grain by-products, etc., and properly balanced for energy, protein, fibers, minerals and vitamins.

Such diets are formulated according to the specific requirements of the target animal mostly by commercial feed mill operations as meal type, pellets or crumbles.

In general commercially prepared feeds are in the form of: 1. complete feeds or rations that provide all the daily required nutrients, 2. concentrate feeds that provide a part of the ration (protein, energy) that are often combined with roughage such as hay or silage, or 3. feed supplements that only provide additional micronutrients, such as minerals and vitamins in the form of meals, pellets or crumbles.

It is an important goal of livestock producers to optimize efficiency of feed conversion of the feedlot diet into edible human food products of high quality, without posing any significant risk to the consumer. A number of approaches have become very common to improve conversion of animal feed into meat, three of the more practical approaches being supplementing the animal feed with (i) hormones, (ii) adrenergic β-agonist compounds or (iii) antimicrobial- and ionophore medicated feed additives. Whereas, the antimicrobial additive approach aims at decreasing populations of pathologic bacteria in the hosts' gastro-intestinal (GI) tract, β-agonist compounds preferentially increase nutrient partitioning to muscle. The two commercially available β-agonist compounds for use in beef cattle production are ractopamine and zilpaterol. Frequently used antimicrobial feed additives in cattle include monensin and tylosin. Melengestrol acetate (MGA) is another orally active feed additive commonly used in feedlot animals that suppresses recurrent estrus and increases growth rate and improves feed efficiency in heifers.

Currently, in most intensive production systems, such a feed lots, cattle feed is mixed and typically distributed 2 to 3 time per day. In practice, all the animals in a single pen receive the same feed at any given meal or feeding. However, animals are grouped in pens based on similar characteristics such as size, sex and age. Each grouping or pen may require different feed requirements based on the aforementioned characteristics.

This is particularly true when there are feed additives that are mixed into the feed. For example, while all cattle may be fed certain feed additives such as monensin and tylosin, only heifers are fed melengestrol acetate (MGA). Further, zilpaterol is only administered to animals during the last 20-40 days prior to slaughter. Therefore, the mixing of different feed compositions for different pens can be a complicated and often laborious process. For example, many feed lots are mixing feed compositions for the cattle requiring zilpaterol 2-3 times per day in every feeding. If the feedlot has pens of heifers, yet another feed composition containing zilpaterol and MGA is required to be prepared several times per day for those particular pens of cattle.

Moreover, distribution of each feed composition to the correct pen adds complexity to the task of feeding the cattle and can be a source of error which requires management attention.

Hence, there is a long felt need for a method of increasing efficiency of beef production in bovine animals that produces, at least, equivalent results to the current feed practices, but that, at the same time, can reduce the complexity of these feeding operations.

Zilpaterol is a known adrenergic β-2 agonist corresponding in structure to Formula (I):

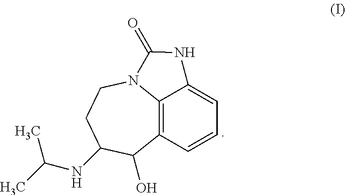 [SMILES: CC(C)NC1CCn2c(=O)[nH]c3cccc(c32)C1O]

The IUPAC name for zilpaterol is 4,5,6,7-tetrahydro-7-hydroxy-6-(isopropylamino) imidazo[4,5,1-jk]-[1]benzazepin-2(1H)-one. The Chemical Abstracts name for zilpaterol is 4,5,6,7-tetrahydro-7-hydroxy-6-[(1-methyl-ethyl)amino]-imidazo[4,5,1-jk][1]benzazepin-2(1H)-one.

Zilpaterol hydrochloride is sold by Merck Animal Health, under the trademark Zilmax®. It is approved in the United States for increased rate of weight gain, improved feed efficiency, and increased carcass leanness in cattle fed in confinement for slaughter during the last 20 to 40 days on feed. The approved inclusion rate of zilpaterol hydrochloride is 6.8 grams/ton (7.5 ppm) in feed on a 90% dry matter basis that is fed continuously as a sole ration in the last 20-40 days of the animal's life prior to slaughter. See NADA No. 141-258.

In U.S. Pat. No. 4,585,770, Frechet et al. discuss compounds, such as zilpaterol, encompassed by a genus characterized as 6-amino-7-hydroxy-4,5,6,7-tetrahydro-imidazo[4,5,1-j-k][1]-benzazepin-2-(1H)-one derivatives and acid addition salts thereof. Fréchet et al. state that such compounds may be used as an active ingredient for inducing antihypertensive and hypotensive activity in a warm-blooded animal.

In U.S. Pat. No. 4,900,735, Grandadam discusses a zootechnical composition comprising zilpaterol and acid addition salts thereof. Grandadam states that such a composition may be used in general to increase the weight of cattle, pigs, sheep, and poultry.

In U.S. Pat. Nos. 5,731,028 and 5,847,124, Chevremont et al. discuss crystallized anhydrous zilpaterol hydrochloride, and particularly crystallized anhydrous zilpaterol hydrochloride wherein less than 5% of the crystals have a size of less than 15 μm, and at least 95% of the crystals have a size of less than 250 μm. According to Chevremont et al., such crystals may be incorporated into animal feed to increase body weight and meat quality.

U.S. Patent Application Publication No. US 2010/0121050 and No. US 2012/0058189 disclose a process for making zilpaterol, salts—and crystalline forms thereof, as well as various administration methods and—regimes for a variety of livestock animals.

Ractopamine hydrochloride is another β-agonist that is currently added to feed in order to increase the growth performance of cattle and swine.

The effects of both ractopamine and zilpaterol on finishing performance, carcass characteristics and meat quality of feedlot steers when fed according to a customary schedule of twice a day feeding of a complete diet is, for example, reported in Avendano-Reyes et al. (J. Animal Sciences 84, 3259-3265, 2006), and Elanco Optaflexx® Research Briefs 3-5, 2011-2012.

Top dress feeding of feed additives to cattle is an alternative to complete feeding programs that increases flexibility to the cattle producer and at the same time reduces labor- and feed-manufacturing costs. This allows farmers to target pens of cattle with unique needs that, in particular, exist in feedlots.

Alternative methods of delivering the zilpaterol and ractopamine feed additives have been developed (Gonzalez et al., Florida Beef Report 2009, 77-82; O'Neill et al., S. African Journal Anim. Science 40, 185-189, 2010; Freedom of Information Summary, Supplemental New Animal Drug Application NADA 141-221).

### SUMMARY OF THE INVENTION

An embodiment of the present invention is a method of improving the efficiency of beef production from a bovine animal which is fed multiple times per day, comprising delivering to the animal an effective dose of zilpaterol in one or more but not all of daily feedings and wherein the zilpaterol is thoroughly mixed into the feed before use.

In an alternative embodiment, the average daily gain (ADG) of the animal is increased.

In an alternative embodiment, the feed efficiency ratio (FE) of the animal is decreased.

In an alternative embodiment, the hot carcass weight of the animal is increased.

In an alternative embodiment, the dressing percentage of the animal is increased.

In an alternative embodiment, the animal is fed complete feedings.

In an alternative embodiment, the animal is kept in confinement.

In an alternative embodiment, the confinement is in a feedlot.

In an alternative embodiment, the concentration of zilpaterol in the feed is from about 6.8 g/ton to about 360 g/ton.

In yet another embodiment, the concentration of zilpaterol in the feed is greater than about 68 g/ton to about 360 g/ton.

In an alternative embodiment, the effective dose of zilpaterol is between 60 to 90 mg/head/day.

In yet another embodiment, the effective dose of zilpaterol is 60 mg/head/day.

In an alternative embodiment, the hot carcass weight of animals treated with zilpaterol is about 30 lb per greater than the hot carcass weight of untreated animals.

Yet another embodiment is a method of improving the efficiency of beef production from a bovine animal which is fed multiple times per day, comprising mixing a Type A medicated article comprising zilpaterol thoroughly into a Type C medicated feed and delivering the Type C medicated feed to the bovine animal in one or more but not all of daily feedings

In an alternative embodiment, the Type A medicated article is mixed into a Type B medicated feed before mixing it into a Type C medicated feed.

In an alternative embodiment, the Type A medicated article comprises 48 g/kilogram zilpaterol hydrochloride.

In an alternative embodiment, the Type C medicated feed comprises an effective dose of zilpaterol hydrochloride of 60-90 mg/head/day.

In an alternative embodiment, the Type C medicated feed is delivered to the bovine animal fed in confinement for slaughter during the last 20-40 days on feed.

In an alternative embodiment, one or more of the variables selected from the group consisting of average daily gain (ADG), feed efficiency (FE), dressing percent and hot carcass weight are improved.

Another embodiment is a cattle feed composition comprising zilpaterol in a concentration of greater than about 75 g/ton to about 180 g/ton.

Another embodiment is a cattle feed composition for bovine comprising zilpaterol in a concentration sufficient to deliver a dose of 60 to 90 mg/head/day of zilpaterol in a single feeding per day, wherein the amount of feed composition delivered to an animal is between 1.0 lb and 26.5 lb on a 90% dry matter basis and the concentration of zilpaterol in the feed is greater than about 75 g/ton to about 180 g/ton.

In an alternative embodiment, the dose of zilpaterol is 60 mg/head/day.

In an alternative embodiment, the zilpaterol is zilpaterol hydrochloride.

In an alternative embodiment, the zilpaterol is adhered to a carrier.

In an alternative embodiment, the carrier is corncob meal.

An additional embodiment is a Type A Medicated Article that comprises greater than about 6.8 g/ton of zilpaterol.

An additional embodiment is a liquid Type B Medicated Feed that comprises 68 to 680 g/ton of zilpaterol.

An additional embodiment is a Type C medicated component feed that comprises greater than about 6.8 g/ton to about 20.4 g/ton of zilpaterol.

In an alternative embodiment, the Type C medicated component feed comprises about 13.6 g/ton of zilpaterol.

An additional embodiment is a Type A Medicated Article that is labeled with instructions to deliver the Type A Medicated Article according to the method of the present invention.

### BRIEF DESCRIPTION OF THE FIGURES

FIG. 1 shows the hot carcass weights of cattle of a control group (without zilpaterol), groups of cattle that received 60, 75 and 90 mg/head/day zilpaterol by component feeding (once per day) and a group fed continuously 6.8 g/ton zilpaterol.

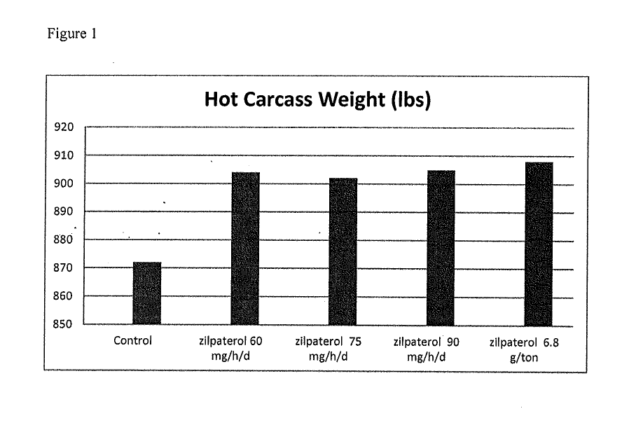

### DEFINITIONS

“Dry matter intake” (DMI) refers to the feed intake, usually per day, expressed in terms of its dry matter content.

“Average daily gain” (ADG) refers to body weight gain (lb)/number of days.

“Feed efficiency ratio” (FE)=DMI/ADG. Improved feed efficiency means a decrease in the ratio of DMI/ADG.

“Hot carcass weight” is the “hot” or unchilled weight in pounds (taken after slaughter and after the hide, head, intestinal tract and internal organs have been removed).

“Dressing percentage” is determined by dividing the hot carcass weight by the live weight, then multiplying by 100.

“Improving the efficiency of beef production” means improvement in one or more of the live growth performance variables of ADG (increasing) and FE (decreasing) or improvement in one or more of the carcass variables of carcass leanness (increasing), dressing percentage (increasing) and hot carcass weight (increasing), of a bovine animal that is fed zilpaterol containing feed compared to a bovine animal that is fed feed without zilpaterol.

“Bovine animal” refers to an animal of genus Bos taurus or Bos indicus, in particular cattle, buffalo, zebu or yak, more in particular cattle, that are raised to produce meat for human food, specifically, cows, bulls, heifers or steers.

“Thoroughly mixed into the feed” means the component (e.g. the zilpaterol) is uniformly distributed through out the feed. Methods are known to one skilled in the art to determine and monitor that the feed is thoroughly mixed (see Herrman et al., MF1172, Kansas State University Agricultural Experimental Station and Cooperative Extension Service, October 1994). “Well mixed feed enhances animal performance and is an essential step in complying with Food and Drug Administration (FDA) Current Good Manufacturing Practices regulation Title 21 C.F.R. 225.30,130).” For example, using the method described in Herrman, samples are taken at 10 predetermined locations or at even intervals during the mixing process. Each sample is analyzed for its content, e.g., zilpaterol content. A coefficient of variation (CV) between the contents of the samples is then calculated.

A CV value of less than 10% in considered excellent.

“Ton” is a unit of measure for mass equal 2000 lb.

“ppm” is an abbreviation of parts per million. ppm is a value that represents the part of a whole number in units of 1/1000000.

“ppm’ is dimensionless quantity, a ratio of 2 quantities of the same unit. For example, a 5 ppm concentration of zilpaterol means 5 mg of zilpaterol per 1 kg of feed. In another example, a 5 ppm concentration of zilpaterol means 5 g of zilpaterol per 1 metric ton (1000 kg) of feed. a 5 ppm concentration of zilpaterol means 4.55 g of zilpaterol per 1 ton (2000 lb) of feed.

“zilpaterol” means 4,5,6,7-tetrahydro-7-hydroxy-6-(isopropylamino) imidazo[4,5,1-jk]-[1]benzazepin-2(1H)-one or any salts or solvates thereof. It also includes salts or solvates of zilpaterol, e.g. the hydrochloride salt.

“Multiple feedings per day” means more than one feeding per day to an animal.

“Effective amount” is an amount of zilpaterol sufficient to improve the efficiency of beef production from a bovine animal.

“ad libitum feeding” means food available at all times with the quantity and frequency of consumption being the free choice of the animal.

“Component feeding” means that a particular component of the feed, especially a feed additive, e.g. zilpaterol, is mixed thoroughly in the feed and delivered to the bovine animal in one or more but not all of the daily feedings.

The following types of Medicated Products are defined in US 21 CFR §558.3)

“Type A Medicated Article”: (previously called Premix) is intended solely for use in the manufacture of another Type A Medicated Article or a Type B or Type C Medicated Feed. It consists of a new animal drug (s) with or without a carrier (e.g., calcium carbonate, rice hull, corn, gluten) with or without inactive ingredients.

“Type B Medicated Feed”: (Type B feed) (previously called Concentrate) is intended solely for the manufacture of other medicated feeds (Type B or Type C). It contains a substantial quantity of nutrients including vitamins and/or minerals and/or other nutritional ingredients in an amount not less than 25 percent of the weight. It is manufactured by diluting a Type A Medicated Article or another Type B Medicated Feed.

“Type C Medicated Feed”: (Type C feed) is intended as the complete feed for the animal or may be fed “top dressed” (added on top of usual ration) on or offered “free-choice” (e.g., supplement) in conjunction with other animal feed. It contains a substantial quantity of nutrients including vitamins, minerals, and/or other nutritional ingredients. It is manufactured by diluting a Type A Medicated Article or a Type B Medicated Feed. A Type C Medicated Feed may be further diluted to produce another Type C Medicated Feed.

Other definitions for selected terms used herein will be found within the description of the invention and apply throughout. Unless otherwise defined, all other scientific and technical terms used herein have the same meaning as commonly understood by individuals who are skilled in the art.

### DETAILED DESCRIPTION OF THE INVENTION

An advantageous method of delivering a beta agonist feed additive to bovine animals has now been developed by the present inventors. This method is easier and more efficient for the user to execute than the existing feeding programs and at the same time affords equivalent efficiency of beef production results. Furthermore this invention relates to products (Type C Feed for bovine animals, Type A medicated articles) that are useful in such methods.

In the Examples below, it is shown when zilpaterol is delivered to cattle once a day in a typical 2× per day feeding program and is thoroughly mixed into the feed, efficiency of beef production results can be obtained that are equivalent to those obtained in the currently used feeding program in the field where the zilpaterol is delivered to the animals in each feeding. This once a day thoroughly mixed method requires fewer feed composition preparations per day. It also offers the advantage that there are fewer distributions of different feed compositions. This reduces the potential error that a feed composition could be delivered to the wrong pen.

Accordingly, in an embodiment the invention provides a method of improving the efficiency of beef production from a bovine animal which is fed multiple times per day, comprising delivering to the animal an effective dose of zilpaterol in one or more but not all of daily feedings and wherein the zilpaterol is thoroughly mixed into the feed before use.

Whereas the favorable efficiency of beef production results can be obtained by a compound with the structure shown in formula I, or any salts or solvates thereof, zilpaterol hydrochloride is preferably used in methods and compositions according to this invention.

In an embodiment the methods and compositions of the present invention are useful for improving the “efficiency of beef production” of a bovine animal.

In a further embodiment, the methods and compositions of the present invention are useful for improving one or more of the efficiency of beef production variables of average daily gain (ADG), feed efficiency (FE), and to dressing percentage and hot carcass weight.

The methods and compositions according to the present invention can advantageously by applied in intensive production systems, such as feedlots, wherein cattle is kept and raised in confinement until slaughter and wherein efficiency of beef production of the animals is a key parameter that determines the economic success of the livestock producer (see also Merck Veterinary Manual, 10^{th} Edition, Management and Nutrition, 2010).

Especially in intensive production systems, such as feedlots, cattle are often fed multiple times or meals per day. In feedlots, over 90% are feed 2-3 times per day (see Vasconcelos, et al. J. ANIM SCI 2007, 85 2772-2781). Therefore, in a preferred embodiment, the method according to the invention comprises improving the efficiency of beef production from cattle, in particular heifers and/or steers that are kept in confinement, such as in a feedlot.

In a more specific embodiment the method according to the invention is used with cattle fed in confinement during the last 20-40 days on feed.

In further embodiments, the bovine animal is fed 2-times day or 3-times per day. The frequency of feedings, however, can be greater than 3 feedings per day.

The exact nutrient composition and formulation of the feedings used herein are not critical to the present invention and can be selected from the compositions and formulations that are currently used in the field. As long as the ingredients are selected according to the nutrient requirements of a particular animal for which the feeding is intended. Such requirements depend, for example, upon age, stage of development of the animal and the sex of the animal. Commonly used cattle feed compositions and formulations are published by the National Academy of Sciences, Nutrient Requirements of Beef Cattle, Appendix Tables 1-19, 192-214, National Academy Press (2000) and Merck Veterinary Manual (10^{th} Edition, 2010, Feeding and Nutritional management of Beef cattle).

In particular, the feedings useful in the practice of the present invention include forages and grain feeds, such as grass and legume forages, crop residues, cereal grains, legume by-products and other agricultural by-products. As used herein, “forages” include the cut aerial portion of a plant material, both monocotyledonous and dicotyledonous, used as animal feed. Examples include, without limitation, orchard grass, timothy, tall fescue, ryegrass, alfalfa, sainfoin, clovers and vetches. With “grain feeds,” are meant the seeds of plants that are fed to bovine animals and may or may not include the outer hull, pod or husk of the seed. Examples include, without limitation, corn, wheat, barley sorghum, triticale, rye, canola, and soya beans.

In beef feedlots, young growing cattle are fed a high energy and high protein feeding to produce marketable beef at a low cost of gain. Mean nutrient contents of feeds commonly used in beef cattle diets, in particular of growing and finishing beef cattle is disclosed in the Merck Veterinary Manual (10^{th} Edition, 2010, Feeding and Nutritional management of Beef cattle, and Vasconcelos et al., J Anim Sci, 85, 2772-2781, 2007).

A high energy diet, in particular, consists of daily feedings that have relatively high grain content. The grain content of feedings to be used herein is typically between 70-90% (w/w), more in particular between 75-85% (w/w).

In a further embodiment the feedings delivered to the bovine animal in a method according to the invention comprises a grain content between 70-90% (w/w), more in particular between 75-85% (w/w).

Favorable crude protein (CP) content of feedings to be used herein are typically between 12-14% (w/w), more in particular between 12.5-13.5% (w/w).

Optimal efficiency of beef production can be achieved when the bovine animals receive feedings with a total daily energy content of 1.37-1.70 MCal/kg net energy for gain (NEg), preferably 1.45-1.60 MCal/kg NEg.

Accordingly, in a preferred method of the invention, the bovine animal receives feed with a total daily CP content of 12-14% (w/w), more in particular between 12.5-13.5% (w/w).

In another preferred method the bovine animal receives feed with a total daily NEg content of 1.37-1.70 MCal/kg, preferably 1.45-1.60 MCal/kg.

In a preferred embodiment the feedings delivered to the animal are complete feedings, i.e. feedings that are balanced as to their nutrient composition. In a complete feeding the roughage and the concentrate ingredients, including the protein, mineral, vitamin and other ingredients are all fed as one mixture. This means that the composition of a complete feeding is such that it provides a balanced diet in each mouthful an animal consumes.

In accordance with the present invention one or more but not all of the multiple feedings per day comprises zilpaterol thoroughly mixed into the feed. Such mixed cattle feed can be produced by specialized commercial feed mills that blend the various raw materials and the feed additives, including zilpaterol, according to the specifications outlined by an animal nutritionist by applying commonly used milling techniques.

Such feed compositions are, preferably, produced by mixing nutritional feed ingredients with a premix comprising the zilpaterol. The premix to be used in the context of the present invention comprises zilpaterol and a suitable carrier or diluent. A preferred premix is a Type A Medicated Article comprising zilpaterol.

Carriers suitable for use to make up the feed composition may include the following: calcium carbonate, alfalfa meal, soybean meal, cottonseed oil meal, linseed oil meal, sodium chloride, cornmeal, cane molasses, urea, bone meal, corncob meal, rice kernel and the like. The carrier promotes a uniform distribution of the active ingredient in the finished feed into which the carrier is blended. It thus performs an important function by ensuring proper distribution of the active ingredient throughout the feed.

Accordingly, a feed composition to be used in a method according to the present invention preferably comprises zilpaterol that is adhered to a carrier, in particular to corncob meal.

The amount of feed consumed in a meal (intake lb/meal) is generally between 0.5 lb/meal and 45 lb/meal. The amount of feed consumed per day (intake pounds/day) is generally in the range of 1 to 90 lb/day.

Therefore, in an embodiment, in order to deliver to the animal zilpaterol in an amount of 60 to 90 mg/head/day, the zilpaterol concentration in the feed (when expressed in grams of zilpaterol per ton of feed on a 90% dry mater basis) is from about 2 g/ton to about 360 g/ton.

In further embodiments, the zilpaterol concentration in the feed is about 6.8 g/ton to about 128 g/ton, preferably 6.8 g/ton to about 20.4 g/ton.

In another embodiment, the range is greater than about 75 g/ton to about 128 g/ton.

In other embodiments, the effective dose of zilpaterol delivered to the animal is between 60 to 90 mg/head/day, preferably, the dose of zilpaterol is 60 mg/head/day.

In an embodiment, the hot carcass weight of animals treated with zilpaterol is about 30 lb per greater than the hot carcass weight of untreated animals.

In again a further embodiment, the present invention also provides cattle feed, such as Type C Medicated Feed that can advantageously be used in a method as described above. Such feed compositions may have the same or similar nutrient composition as those commonly used in the field by cattle producers, for example, as described in more detail above, but are distinguished therefrom by a higher concentration of zilpaterol (higher than 6.8 g. zilpaterol per ton feed).

Such a cattle feed composition is characterized in that it comprises zilpaterol in a concentration of about 2 g/ton to about 360 g/ton. In another embodiment, the range is about 6.8 g/ton to about 128 g/ton. In another embodiment, the range is greater than about 75 g/ton to about 128 g/ton. In another embodiment, the range is about 6.8 g/ton to about 20.4 g/ton.

The present invention also provides a Type A medicated article comprising zilpaterol, preferably comprising 48 g/kilogram zilpaterol hydrochloride intended for use in the manufacture of medicated animal feed, such as a Type C medicated feed. Such Type A Medicated Article is labeled with instructions to deliver the Type A Medicated Article as component feeding. This means, the label on the Type A Medicated Article contains instructions to deliver to the animal an effective dose of zilpaterol in one or more but not all of daily feedings and wherein the zilpaterol is thoroughly mixed into the feed before use. Such label can be printed on the packaging of such Type A Medicated Article, e.g. on the bag or container, can be used as an adhesive label on the packaging or container, or alternatively the label can be delivered together with packaging on the package insert or other papers of the Type A Medicated Article.

The following paragraphs describe embodiments of the present invention that also provides a Type A Medicated Article comprising zilpaterol for delivery to cattle prior to slaughter. In an embodiment, the cattle are kept in confinement prior to slaughter.

In yet another embodiment, the confinement is in a feedlot.

In an embodiment, Type A Medicated Article comprising zilpaterol is available. The Type A Medicated Article contains 4.8% zilpaterol hydrochloride (48 grams per kilogram). In a further embodiment, the Type A Medicated Article contains ground corncobs, surfactant and binder as an inert ingredients. In another embodiment, the Type A Medicated Article is diluted in a suitable carrier before addition to the final feed. In an alternative embodiment, the Type A Medicated Article is diluted to with additional appropriate feed to form of the Type C Medicated Feed. In an alternative embodiment, the Type A Medicated Article is first mixed to form a liquid Type B feed which is then subsequently used to prepare the Type C Medicated Feed.

Before feeding, the Liquid Type B Medicated Feed is thoroughly mixed with other feed materials to make a Type C Medicated Feed. Examples of addition rates for component feeds are shown in the following table.

| | Pounds of Liquid Type B | Resulting Zilpaterol | | |
|---|---|---|---|---|
| Zilpaterol in | Feed To Add Per Ton To | Concentration in | Type C Medicated | Zilpaterol |
| Liquid Type | Make Type C Medicated | Type C Medicated | Component Feed | Consumed Daily |
| B Feed | Component Feed | Component Feed¹ | Consumption¹ | by Each Animal |
| g/ton | lb/ton | g/ton | lb/head/day | mg/head/day |
| 68 | 200 | 6.8 | 17.6 | 60 |
| 68 | 200 | 6.8 | 26.5 | 90 |
| 340 | 80 | 13.6 | 8.8 | 60 |
| 340 | 80 | 13.6 | 13.3 | 90 |
| 680 | 60 | 20.4 | 5.9 | 60 |
| 680 | 60 | 20.4 | 8.8 | 90 |

¹Based on 90% dry matter basis

In an embodiment, to prepare zilpaterol for administration by component feeding, thoroughly mix Type A Medicated Article comprising zilpaterol in a ton of appropriate feed ingredients or diluents according to the table below to obtain the proper concentration in the Type C Medicated Feed (minimum 6.8 g/ton). The following table gives examples of how some Type C Medicated Feed concentrations can be prepared.

| Pounds of zilpaterol containing | Resulting Zilpaterol | | |
|---|---|---|---|
| Type B Medicated Article (0.34 | Concentration in | Type C Medicated | Zilpaterol |
| g/lb)¹ to Add Per Ton To Make | Type C Medicated | Component Feed | Consumed Daily |
| Type C Medicated Component Feed | Component Feed² | Consumption | by Each Animal |
| Component Feed | g/ton | lb/head/day | mg/head/day |
| 20 | 6.8 | 17.6 | 60 |
| 20 | 6.8 | 26.5 | 90 |
| 40 | 13.6 | 8.8 | 60 |
| 40 | 13.6 | 13.3 | 90 |
| 60 | 20.4 | 5.9 | 60 |
| 60 | 20.4 | 8.8 | 90 |

¹Throughly mix 31.2 lb of Type A Medicated Article in a ton of appropriate feed ingredients or diluents to make one ton of Type B Medicated Feed (0.34 g/lb).
²Based on 90% dry matter basis

The above Feeds are fed during one or more but not all of the daily feedings to cattle fed in confinement for slaughter containing no less than 6.8 g/ton for the last 20 to 40 days on feed to provide 60 to 90 mg zilpaterol hydrochloride per head per day. The maximum amount of Type C Medicated Feed in a single feeding must not exceed 17.6 lb. to 26.5 lb. to provide 60 to 90 mg/head/day, respectively.

Accordingly, in an embodiment the invention provides a method of improving the efficiency of beef production from a bovine animal which is fed multiple times per day, comprising mixing a Type A Medicated Article comprising zilpaterol thoroughly into a Type C Medicated Feed and delivering the Type C Medicated Feed to the bovine animal in one or more but not all of daily feedings.

In a more specific embodiment, the Type A Medicated Article is mixed into a Type B Medicated Feed before mixing it into a Type C Medicated Feed.

In a further embodiment the Type A Medicated Article comprises 48 g/kilogram zilpaterol hydrochloride.

In embodiment the Type C Medicated Feed comprises an effective dose of zilpaterol hydrochloride of 60-90 mg/head/day.

In another embodiment the Type C Medicated Feed is delivered to the bovine animal fed in confinement for slaughter during the last 20-40 days on feed.

In a further embodiment one or more of the variables selected from the group consisting of average daily gain (ADG), feed efficiency (FE), dressing percent and hot carcass weight are increased.

### Example 1

### Comparison of Component Feeding at Various Doses (60, 75 and 90 mg/Head/Day) to Continuous Feeding at 6.8 g/Ton

A multi-center, randomized complete block design was used. Four study sites enrolled 800 to 1000 head of cattle each. Cattle were randomized to 10 blocks of 80 to 100 head based on body weight, then penned in groups of 16 to 20 head per pen.

The pen served as the experimental unit. Treatment groups were as described below in Table 1:

TABLE 1
Experimental Treatment Groups

| Group No. | Treatment Group |
|---|---|
| 1 | Control (no Zilmax ®) |
| 2 | Zilmax ® component feeding - 60 mg/head/day |
| 3 | Zilmax ® component feeding - 75 mg/head/day |
| 4 | Zilmax ® component feeding - 90 mg/head/day |
| 5 | Zilmax ® fed continuously in a Type C feed - |
| | 6.8 g/ton (90% DMB) |

Commercial Zilmax® Type A Medicated Article containing 4.8% (21.77 grams per pound) of zilpaterol hydrochloride was used in the studies.

Finishing steers that were visually estimated to be approximately 58 days from finish were used in the studies. Each site enrolled 800 to 1000 head of cattle. Cattle were randomized to 10 blocks of 80 to 100 head and, within block, to treatment group based on body weight. Study pens consisted of 16 to 20 head per pen.

The study was broken into three periods: acclimation, Zilmax® treatment and withdrawal. Between study sites, the number of acclimation days was variable but the Zilmax feeding period was 20 days at all study sites. The withdrawal period ranged from 3 to 5 days across the sites. More than one start time was allowed to accommodate enrollment of the study animals. If blocks were initiated at different times, blocks may have been acclimated for different lengths of time. The minimum length of the acclimation period was 31 days.

During the study, animals were housed in typical feedlot drylot (dirt floored) pens by block and treatment group. Pens provided adequate pen space per animal. Feed bunks provided 9 to 10 inches of space per animal to simulate commercial feeding practices.

All animals were permitted ad libitum feed consumption according to standard study site procedure. Feed delivery occurred twice daily. Daily feed issue was recorded on a per pen basis from the start of the acclimation period and until final weights were measured before shipment for harvest. Feed batching and delivery were documented.

A component feeding method was tested in the studies by thoroughly mixing the daily zilpaterol dose in the first feeding of the Type C feed prior to delivery to the animal for Groups 2, 3 and 4. Homogeneity of Zilmax® mixing in the Type C feed was demonstrated by the study site prior to initiation of the Zilmax® feeding period.

Feedstuffs fed in the study were typical for the geographical location of the study sites and diets met the nutrient requirements of the study animals. Diets were formulated using typical feedlot diet procedures. At each study site, all cattle were fed a single formulation of a high concentrate Type C complete feed once the Zilmax® feeding period of the study started. The diets contained monensin and tylosin at 30 g/t and 9 g/t, respectively, on a 90% dry matter basis (DMB) throughout the study. No other feed additives were included in the diets. Feed bunk management (e.g., increases and decreases in daily feed offering, unscheduled feed removals) were according to the standard practice at the study sites.

The daily component feed amount (i.e., the first feeding each day of the Type C feed) was set at approximately 10 lb of feed dry matter (DM) per head to simulate commercial practice for component feeding. Therefore, all study pens were fed approximately 10 lb of DM per head in the first feeding of the day. Control steers were fed the Type C feed that contained no Zilmax. The component feeding treatment groups were fed the Type C feed that contained 60, 75, or 90 mg/head/day.

All cattle in the control and three component feeding treatments were fed the non-medicated Type C complete feed (i.e., without Zilmax®) in a second feeding each day. Steers in the 6.8 g/ton Zilmax® treatment group received the Type C complete feed containing this level of Zilmax® in both feedings each day during the 20-day Zilmax® feeding period.

Zilmax® Type A Medicated Article was diluted prior to addition to the Type C feed by use of a micro-ingredient machine or preparation of a Type B feed. During the Zilmax® feeding period, daily Type C feed samples were taken from each treatment group and composited on an approximately weekly basis prior to zilpaterol hydrochloride and moisture assays.

Individual animal weights were measured prior to the start of the Acclimation period for use in randomization of animals to treatment groups and pens. The Acclimation period was used to stabilize feed intake and behavioral aspects of the newly sorted pen groups of cattle prior to feeding Zilmax®. Individual weights were measured and unconsumed feed was removed from the feed bunks and weighed on the day of initiation of Zilmax® feeding. After a 20-day Zilmax® feeding period, unconsumed feed was removed from the feed bunks, weighed, and discarded. All animals in the five treatment groups were fed a non-medicated feed (i.e., without Zilmax®) for a minimum 3 day withdrawal period prior to harvest. Unconsumed feed remaining in the feed bunks was removed and weighed on the day of final body weight measurement. Cattle were handled throughout the study using procedures which were typical of commercial practices. Cattle were exposed to ambient weather conditions during the studies. Feed was not restricted prior to weighing or harvest.

The primary variables in this study were average daily body weight gain (ADG) and feed efficiency (FE) calculated for the Zilmax® feeding period of the study (Day 0 through end of withdrawal period) and for the entire study (start of the acclimation period to end of withdrawal period).

ADG was calculated as a pen average from the ADG of individual animals within the pen using the equation: ADG=(final body weight−Day 0 body weight)/(number of days between the body weight measurements). Only animals that completed the entire study were used in the calculation of ADG.

Dry matter intake (DMI) for each pen of animals was calculated by correcting daily as-fed feed issues and any feed weighback weights to a 100% dry matter basis using the moisture concentrations determined at the zilpaterol analytical laboratory on the diet samples. Feed weighback dry matter was then subtracted from the feed issue dry matter to calculate total feed dry matter consumed. Daily dry matter intake per pen per day was calculated by dividing the total feed dry matter consumed by the animals in the pen by the total number of head days for the pen.

FE for each pen was calculated using the equation: FE=DMI/ADG.

ADG, DMI and FE were calculated for the two study periods (start of the acclimation period or Day 0 to the end of withdrawal).

DMI was calculated for the two study periods (start of the acclimation period or Day 0 to the end of the withdrawal period). The following carcass data were collected or calculated: marbling score, USDA quality grade or equivalent, hot carcass weight, ribeye area, backfat thickness (preliminary yield grade and adjusted preliminary yield grade), and USDA yield grade or equivalent. Dressing percent was calculated.

Variables continuous in nature were subjected to the analysis described for the primary outcome variables. Outcomes evaluated included: initial body weight, final body weight, weight gain (Day 0 to final date), ADG (acclimation period, initial date to final date), ADG (shrunk, Day 0 to final date and initial date to final date), DMI (acclimation, initial date to final date), as fed intake (Zilmax® period), carcass gain (Day 0 to final date), dressing percent, hot carcass weight, marbling score, fat depth, ribeye area, percent KPH and calculated yield grade.

Comparisons were evaluated with Control versus each of the treatment groups. Comparisons of Group 5 (the 6.8 g/ton) versus each of the component feeding levels (60, 75 and 90 mg/head/day) were also made.

### Secondary (Carcass) Variables:

The following carcass data were collected or calculated: marbling score, USDA quality grade or equivalent, hot carcass weight, ribeye area, backfat thickness, calculated yield grade, kidney-pelvic-heart fat percentage, and USDA yield grade or equivalent. The Canadian study site CBGA quality and yield grades were converted to USDA equivalents prior to analysis. Dressing percent was calculated.

The results of the statistical analysis of the primary variables are summarized in Table 2 below.

TABLE 2
Summary of the statistical analysis of the primary variables
(least squares means and standard error of the mean)

| | | 60 | 75 | 90 | | | |
|---|---|---|---|---|---|---|---|
| Variable | Control | mg/hd/day | mg/hd/day | mg/hd/day | 6.8 g/ton | SEM^{(a)} | P-value^{(b)} |
| Number of Head | 747 | 752 | 754 | 750 | 755 | — | — |
| Number of Pens | 40 | 40 | 40 | 40 | 40 | — | — |
| Initial body | 1187 | 1189 | 1187 | 1188 | 1189 | 21.2061 | 0.9982 |
| weight, lb/head | | | | | | | |
| Final body | 1443 | 1463 | 1459 | 1461 | 1467 | 15.7317 | 0.0923 |
| weight, lb/head | | | | | | | |

Acclimation Period (31 to 59 days in length)

| | | | | | | | |
|---|---|---|---|---|---|---|---|
| ADG, | 4.05 | 4.16 | 4.19 | 4.22 | 4.17 | 0.3778 | 0.5554 |
| lb/head/day | | | | | | | |
| DMI, lb/head/day | 22.48 | 22.60 | 22.61 | 22.61 | 22.79 | 1.1014 | 0.5234 |
| FE (Feed / Gain) | 5.74 | 5.61 | 5.58 | 5.50 | 5.68 | 0.4303 | 0.4961 |

Zilmax and Withdrawal Periods (23 to 25 days in length)

| | | | | | | | |
|---|---|---|---|---|---|---|---|
| ADG, | 3.55 | 4.13*^{(c)} | 3.99* | 3.97* | 4.23* | 0.5612 | 0.0144 |
| lb/head/day | | | | | | | |
| DMI, lb/head/day | 23.15 | 22.57* | 22.70* | 22.39* | 22.87 | 1.1825 | 0.0445 |
| FE (Feed / Gain) | 7.38 | 5.89* | 5.99* | 6.10* | 5.96* | 0.7582 | 0.0013 |

Entire Study (55 to 82 days in length)

| | | | | | | | |
|---|---|---|---|---|---|---|---|
| ADG, | 3.88 | 4.14* | 4.12* | 4.13* | 4.19* | 0.4057 | <0.0001 |
| lb/head/day | | | | | | | |
| DMI, lb/head/day | 22.83 | 22.71 | 22.75 | 22.65 | 22.93 | 0.8507 | 0.5136 |
| FE (Feed / Gain) | 6.04 | 5.64* | 5.65* | 5.62* | 5.64* | 0.4299 | <0.0001 |

^{(a)}SEM = standard error of the mean
^{(b)}Overall treatment effect P-value
^{(c)}* = versus control, P < 0.05

Zilmax and Withdrawal Period:

A statistically significant treatment effect was detected for ADG, DMI and FE during the Zilmax period (P<0.05). ADG values were significantly higher in the treated groups compared to the control group. No differences between the component feeding groups and the continuous feeding group were detected. DMI was significantly lower in the component feeding groups compared to the control group. There was no significant difference in DMI between the control group and the continuous feeding group. FE values were significantly lower in the treated groups compared to the control group. No differences between the component feeding groups and the continuous feeding group were detected.

Acclimation to End of Withdrawal:

A statistically significant treatment effect was detected for ADG and FE over the entire study period (P<0.05). ADG values were significantly higher in the treated groups compared to the control group. No differences between the component feeding groups and the continuous feeding group were detected. FE values were significantly lower in the treated groups compared to the control group. No differences between the component feeding groups and the continuous feeding group were detected. No differences among the treatment groups were detected in DMI (P>0.51)

### Secondary (Carcass) Variables:

The results of the statistical analysis of the carcass variables are summarized in Table 3 below.

Dressing percentage, hot carcass weight, marbling score, USDA yield grade score, rib eye area, and calculated yield grade were significantly affected by treatment (P<0.05). Dressing percentage, hot carcass weight, and rib eye area were significantly higher in the Zilmax treated groups compared to the control group and no differences between the component feeding groups and the continuous feeding group were detected. Marbling score, USDA yield grade score, and calculated yield grade values were significantly lower in the Zilmax treated groups compared to the control group and no differences between the component feeding groups and the continuous feeding group were detected. There were no significant differences (P>0.19) among the treatment groups in USDA quality grade, backfat thickness, or kidney-pelvic-heart fat percentage.

TABLE 3
Summary of Secondary (Carcass) Variable Analysis by Treatment Group

| | | 60 | 75 | 90 | 6.8 | | |
|---|---|---|---|---|---|---|---|
| Variable | Control | mg/hd/day | mg/hd/day | mg/hd/day | g/ton | SEM^{(a)} | P-value^{(b)} |
| Number of Head^{(c)} | 648 | 652 | 654 | 651 | 656 | — | — |
| Number of Pens^{(c)} | 35 | 35 | 35 | 35 | 35 | — | — |
| Dressing % (using final | 62.45 | 63.89* | 64.00* | 64.06* | 64.08* | 0.9348 | <0.0001 |
| body weight shrunk 4%) | | | | | | | |
| Hot Carcass Weight, | 872 | 904* | 902* | 905* | 908* | 12.8513 | <0.0001 |
| lb/head | | | | | | | |
| Marbling Score^{(d)} | 477 | 453* | 460* | 456* | 457* | 28.0200 | <0.0001 |
| USDA Yield Grade | 2.42 | 2.19* | 2.19* | 2.18* | 2.19* | 0.3326 | 0.0154 |
| Backfat Thickness, | 0.52 | 0.49 | 0.48 | 0.49 | 0.49 | 0.05127 | 0.1957 |
| inches | | | | | | | |
| Ribeye Area, sq. inches | 14.00 | 15.14* | 15.07* | 15.20* | 15.14* | 0.1877 | <0.0001 |
| Kidney-Pelvic-Heart fat, % | 2.27 | 2.27 | 2.26 | 2.26 | 2.27 | 0.2347 | 0.6884 |
| Calculated Yield Grade | 3.14 | 2.81* | 2.82* | 2.79* | 2.83* | 0.1079 | 0.0003 |

^{(a)}SEM = standard error of the mean
^{(b)}Overall treatment effect P-value
^{(c)}* = versus control, P < 0.05
^{(d)}Site 1 only provided data from 25 of the 50 study pens due to an error by site personnel. Thus, for carcass variables, the total number of pens was 35 per treatment group and the number of head per treatment was 648, 652, 654, 651, and 656 for the Control, 60 mg/hd/day, 75 mg/hd/day, 90 mg/hd/day and 6.8 g/ton treatment groups, respectively.
^{(e)}Marbling score definitions: <300 = standard, 300-399 = select, 400-699 = choice, ≧700 = prime.

1. A method of improving the efficiency of beef production from a bovine animal which is fed multiple times per day, comprising delivering to the animal an effective dose of zilpaterol in one or more but not all of daily feedings and wherein the zilpaterol is thoroughly mixed into the feed before use.

2. The method of claim 1, wherein average daily gain (ADG) of the animal is increased.

3. The method of claim 1, wherein feed efficiency ratio (FE) of the animal is decreased.

4. The method of claim 1, wherein hot carcass weight of the animal is increased.

5. The method of claim 1, wherein dressing percentage of the animal is increased.

6. The method of claim 1, wherein the animal is fed complete feedings.

7. The method of claim 1, wherein the animal is kept in confinement.

8. The method of claim 7, wherein the confinement is in a feedlot.

9. The method of claim 1, wherein the concentration of zilpaterol in the feed is from about 6.8 g/ton to about 360 g/ton.

10. The method of claim 9, wherein the concentration of zilpaterol in the feed is greater than about 68 g/ton to about 360 g/ton.

11. The method of claim 1, wherein the effective dose of zilpaterol is between 60 to 90 mg/head/day.

12. The method of claim 11, wherein the effective dose of zilpaterol is 60 mg/head/day.

13. The method of claim 1, wherein the hot carcass weight of animals treated with zilpaterol is about 30 lb per greater than the hot carcass weight of untreated animals.

14. A method of improving the efficiency of beef production from a bovine animal which is fed multiple times per day, comprising mixing a Type A medicated article comprising zilpaterol thoroughly into a Type C medicated feed and delivering the Type C medicated feed to the bovine animal in one or more but not all of daily feedings

15. The method of claim 14, wherein the Type A medicated article is mixed into a Type B medicated feed before mixing it into a Type C medicated feed.

16. The method of claim 14, wherein the Type A medicated article comprises 48 g/kilogram zilpaterol hydrochloride.

17. The method of claim 14, wherein the Type C medicated feed comprises an effective dose of zilpaterol hydrochloride of 60-90 mg/head/day.

18. The method of claim 14, wherein the Type C medicated feed is delivered to the bovine animal fed in confinement for slaughter during the last 20-40 days on feed.

19. The method of claim 14, wherein one or more of the variables selected from the group consisting of average daily gain (ADG), feed efficiency (FE), dressing percent and hot carcass weight are improved.

20. A cattle feed composition comprising zilpaterol in a concentration of greater than about 75 g/ton to about 180 g/ton.

21. The cattlefeed composition of claim 20 comprising zilpaterol in a concentration sufficient to deliver a dose of 60 to 90 mg/head/day of zilpaterol in a single feeding per day, wherein the amount of feed composition delivered to an animal is between 1.0 lb and 26.5 lb on a 90% dry matter.

22. The cattle feed composition of claim 21, wherein the dose of zilpaterol is 60 mg/head/day.

23. The cattle feed composition of claim 21, wherein the zilpaterol is zilpaterol hydrochloride.

24. The cattle feed composition of claim 21, wherein the zilpaterol is adhered to a carrier.

25. The cattle feed composition of claim 24, wherein the carrier is corncob meal.

26-29. (canceled)
